(19) United States
(12) Patent Application Publication
(10) Pub. No.: US 2022/0307964 A1
(43) Pub. Date: Sep. 29, 2022

(54) METHOD FOR DETERMINING HYDRAULIC PARAMETERS AND WATER INFLOW IN EROSION STAGE OF GRAVEL SOIL

(71) Applicants: Bin Zhu, Guilin, Guangxi Zhuang Autonomous Region (CN); Chuandai Lin, Guilin, Guangxi Zhuang Autonomous Region (CN); Jun Chen, Guilin, Guangxi Zhuang Autonomous Region (CN); Juyan Chen, Guilin, Guangxi Zhuang Autonomous Region (CN); Xianyu Deng, Guilin, Guangxi Zhuang Autonomous Region (CN); Bo Li, Guilin, Guangxi Zhuang Autonomous Region (CN)

(72) Inventors: Bin Zhu, Guilin, Guangxi Zhuang Autonomous Region (CN); Chuandai Lin, Guilin, Guangxi Zhuang Autonomous Region (CN); Jun Chen, Guilin, Guangxi Zhuang Autonomous Region (CN); Juyan Chen, Guilin, Guangxi Zhuang Autonomous Region (CN); Xianyu Deng, Guilin, Guangxi Zhuang Autonomous Region (CN); Bo Li, Guilin, Guangxi Zhuang Autonomous Region (CN)

(21) Appl. No.: 17/458,604

(22) Filed: Aug. 27, 2021

(30) Foreign Application Priority Data
Mar. 26, 2021 (CN) 202110326321.0

(51) Int. Cl. G01N 15/08; G01V 9/00; G01N 33/24

(52) U.S. Cl. CPC G01N 15/0826; G01V 9/00; G01N 33/24

(57) ABSTRACT

The invention discloses method for determining hydraulic parameters and water inflow in the erosion stage of gravel soil, comprising: calculate the soil particle content P and the soil porosity n of each grade of particle size a, and draw the PSD curve of each grade of particle size and the soil particle content P of each grade of particle size and the PSD curve cluster of each grade of particle size and the soil particle content P of each grade of particle size in each erosion stage; calculate the equivalent diameter Dₕ of the soil particle, and calculate the minimum equivalent pore diameter d₀ of the soil particle; calculate the critical hydraulic gradient i_{cr} of particle erosion at each stage; calculate the permeability coefficient kₕ; calculate the seepage flow velocity ν and the total seepage flow Q.

### BACKGROUND OF THE INVENTION

### 1. Field of the Invention

The invention relates to the field of geological and geotechnical engineering, in particular to a method for determining hydraulic parameters and water inflow in the erosion stage of gravel soil.

### 2. Description of the Related Art

Among the many factors that affect the stability of foundation pit excavation and the safety of karst collapse, the infiltration and erosion of gravel soil has attracted widespread attention. Especially in karst areas, the bimodal gravel soil with missing middle grain size or wide-graded gravel soil is an important factor for the formation of rock-soil structures and the formation of karst collapse.

There have been many studies in the existing technologies aimed at the problem of infiltration and corrosiveness such as the loss of fine particles and piping due to the particle distribution characteristics of gravel soil after being immersed in water. Different from fluid soil erosion, in the case of piping seepage erosion and small hydraulic gradients, the seepage erosion of the soil and its stability problems occur. The researches of Sherard, Mace, Indraratna and Radampola have many research results from the physical mechanism of osmotic erosion, experimental methods to evaluation methods. In China, the researches of Liu Jie, Xie Dingsong, Mao Changxi, etc. are representative, especially for the summary of the wide-graded gravel soil classification evaluation model.

Since the seepage flow of rock and soil directly affects the water inrush of foundation pit or dam engineering, it is particularly important to determine the hydraulic characteristic parameters of rock and soil under different erosion conditions. Existing research and related standards are difficult to quantitatively evaluate and define the seepage stability problem from seepage erosion to water inrush, that is, the problem of water inflow is difficult to determine, which leads to sudden safety problems. Therefore, it is necessary to provide a method for determining hydraulic parameters and water inflow in the erosion stage of gravel soil.

### SUMMARY OF THE INVENTION

The embodiment of the invention provides a method for determining hydraulic parameters and water inflow in the erosion stage of gravel soil, which can solve the problem that existing research and related standards are difficult to quantitatively evaluate and define the seepage stability from seepage erosion to water inrush, that is, the problem of water inflow is difficult to determine, which leads to sudden safety problems.

The invention provides a method for determining hydraulic parameters and water inflow in the erosion stage of gravel soil, comprising the following steps:

calculate the soil particle content P and the soil porosity n of each grade of particle size according to the principle of graded erosion, and draw the PSD curve of each grade of particle size and the soil particle content P of each grade of particle size and the PSD curve cluster of each grade of particle size and the soil particle content P of each grade of particle size at each erosion stage;

calculate the equivalent diameter Dₕ of the soil particle according to the average particle diameter Dⱼ in a certain two particle size range in the PSD curve, and calculate the minimum equivalent pore diameter d₀ of the soil particle according to the equivalent diameter Dₕ;

calculate the critical hydraulic gradient i_{cr} of particle erosion at each stage according to the soil particle content P of each grade of particle size;

calculate the permeability coefficient kₕ according to the soil porosity n and the geometric parameter values of the soil particle in the PSD curve cluster;

calculate the seepage flow velocity ν and the total seepage flow Q according to the permeability coefficient kₕ.

Preferably, the method for calculating the soil particle content P and the soil porosity n of each grade of particle size is:

calculate the soil particle content Pⱼ⁽ⁱ⁾ of the j-th grade of particle size in the (1+1) state and the soil porosity n⁽ⁱ⁺¹⁾ updated to the (i+1) state according to the following formula (1):

$$\begin{matrix}{P}_{j}^{( i + 1 )} = \{ \begin{matrix}\frac{{P}_{j}^{( i )} - S}{100 - S} \times 100 \% & ( S \leq {P}_{j}^{( i )} < {P}_{x} ) \\ 0 & ( {P}_{j}^{( i )} < S < {P}_{x} )\end{matrix} & ( 1 a ) \\ {n}^{( i + 1 )} = {n}^{( i )} + ( 1 - {n}^{( i )} ) S & ( 1 b )\end{matrix}$$

in the formula, Pⱼ⁽ⁱ⁾ is the soil particle content of the j-th grade of particle size in the state (i); nⁱ is the soil porosity in the state (i); S is the degree of osmotic erosion, that is, the percentage of the mass of the soil particle smaller than a certain size that are washed away and eroded to the mass of the original soil particle; Pₓ is the content of fine particles, that is, the percentage of soil particles loss in the total soil mass.

Preferably, the method for calculating the minimum equivalent pore diameter d₀ of the soil particle according to the equivalent diameter Dₕ is:

calculate the equivalent diameter Dₕ of the soil particle according to the following formula (2):

$${D}_{h} = \frac{1}{Σ \frac{Δ {S}_{j}}{{D}_{j}}} \qquad ( 2 )$$

calculate the minimum equivalent pore diameter d₀ according to the following formula (3):

$${d}_{0} = 2.67 \frac{n}{1 - n} \frac{{D}_{h}}{α} \qquad ( 3 )$$

in the formula, Dⱼ is the average particle size of the soil particle with a size grade between j₁ and j₂; ΔSⱼ is the ratio of the weight of the j-th grade of particle size to the total weight of the sample; n is the porosity; α is the shape factor of the particle.

Preferably, the method for calculating the critical hydraulic gradient i_{cr} of particle erosion at each stage is:

calculate the critical hydraulic gradient (i_{cr}) according to the following formula (4):

$${( {i}_{α} )}_{j} = \frac{0.85 {d}_{j}}{{P}_{j} {d}_{85}} ( 1 - n ) ( s - 1 ) \qquad ( 4 )$$

in the formula, (i_{cr}) j is the critical hydraulic gradient of the j-th grade of particle erosion; s is the relative density, that is, the density of the overall soil density relative to the density of the water body; d₈₅ is the particle size of the particle whose loss of soil particles accounts for 85% of the total soil mass; dⱼ is the j-th grade of particle size that is eroded from the soil; pⱼ is the particle content of the j-th grade of particle size.

Preferably, the method for calculating the permeability coefficient kₕ is:

calculate the permeability coefficient kₕ according to the following formula (5):

$${k}_{k} = 3.5 \times {10}^{- 4} \frac{{e}^{3}}{1 + e} \frac{{γ}_{w}}{{μ}_{w}} {d}_{10}^{2.32} {C}_{α}^{0.6} \qquad ( 5 )$$

in the formula, e is the void ratio, calculated from the soil porosity e=n/(1−n); μw, is the dynamic viscosity coefficient of water; γ_{w} is the weight of water; d₁₀ is the particle size of the particle whose loss of soil particles accounts for 10% of the total soil mass; Cᵤ is the coefficient of nonuniformity.

Preferably, the method for calculating the seepage flow velocity ν and the total seepage flow Q is:

calculate the seepage velocity ν of the soil particle according to the following formula (6):

v=Kₕ*i_{cr} (6)

calculate the total seepage flow Q of the soil particle according to the following formula (7):

Q=n·ν·A (7)

in the formula, ν is the seepage velocity; i_{cr} is the critical hydraulic gradient; Q is the total seepage flow; A is the area; n is the soil porosity.

Compared with the prior art, the advantages of the invention are:

The method of the invention calculates the dynamic geometric parameters and the changed critical hydraulic gradient and permeability coefficient through the moving PSD curve under the condition of gravel soil graded erosion, and then calculates the seepage velocity and the water inflow by the Darcy formula, so as to obtain the rock and soil hydraulic characteristic parameters and the water inrush, which makes it possible to calculate the total seepage flow in the event of seepage erosion, and inversely deduce the degree of gravel soil erosion and dangerous conditions, so that corresponding measures can be taken to control and protect them, so as to avoid accidents. It is worthy of promotion.

### BRIEF DESCRIPTION OF THE DRAWINGS

In order to explain the embodiments of the invention or the technical solutions in the prior art more clearly, the drawings that need to be used in the description of the embodiments or the prior art will be briefly introduced hereinafter. Obviously, the drawings in the following description are only some embodiments of the invention. For those of ordinary skill in the art, other drawings can be obtained based on these drawings without creative efforts.

FIG. 1 is a flow chart of the method according to the invention;

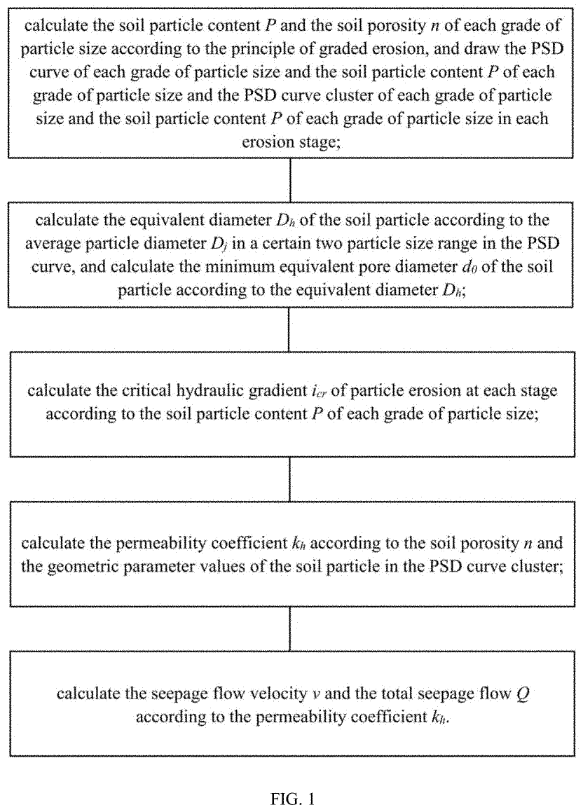

FIG. 2 is a PSD curve of the particle size distribution of the soil particle of three different soil types according to the invention;

FIG. 3 is a PSD curve of each grade of particle size and the soil particle content P of each grade of particle size according to the invention;

FIG. 4 is a PSD curve cluster of the gravel soil S2 according to the invention at each erosion stage;

FIG. 5 is a PSD curve cluster of the gravel soil S3 according to the invention at each erosion stage;

FIG. 6 is a block diagram of calculation of critical hydraulic gradient in the process of particle graded erosion according to the invention;

FIG. 7 is a diagram showing the relationship between the critical hydraulic gradient and each grade of particle size of gravel soil according to the invention;

FIG. 8 is a diagram showing the relationship between the critical hydraulic gradient of gravel soil and the percentage of particles according to the invention;

FIG. 9 is a diagram showing the relationship between geometric parameters and permeability coefficient of the gravel soil S2 under different erosion degrees according to the invention;

FIG. 10 is a diagram showing the relationship between geometric parameters and permeability coefficient of the gravel soil S3 under different erosion degrees according to the invention;

FIG. 11 is a diagram showing the change of the seepage flow velocity ν and the water inflow Q in the low value area of the critical hydraulic gradient i_{cr} at different stages according to the invention;

FIG. 12 is a diagram showing the change of the seepage flow velocity ν and the water inflow Q in the high value area of the critical hydraulic gradient i_{cr} at different stages according to the invention;

### DETAILED DESCRIPTION OF THE PREFERRED EMBODIMENTS

The technical solutions in the embodiments of the invention will be described clearly and completely hereinafter with reference to the drawings 1 to 11 in the embodiments of the invention. Obviously, the described embodiments are only a part of the embodiments of the invention, rather than all the embodiments. Based on the embodiments of the invention, all other embodiments obtained by those of ordinary skill in the art without creative efforts shall all fall within the protection scope of the invention.

The invention provides a method for determining hydraulic parameters and water inflow in the erosion stage of gravel soil.

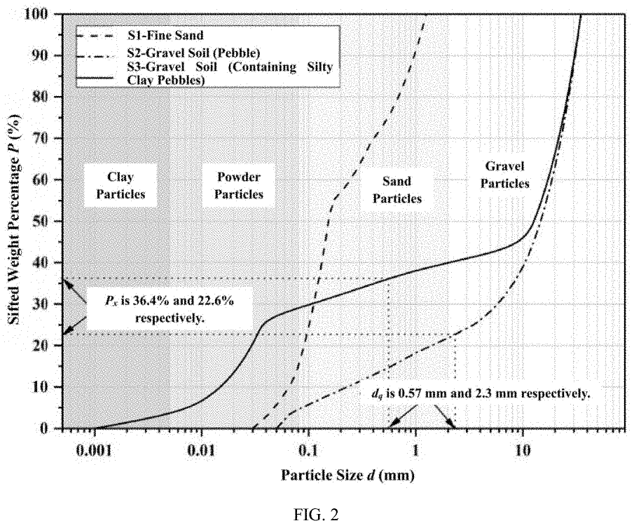

Before implementing the method, it is necessary to draw the PSD curve of the particle size distribution of the three different soil types as shown in FIG. 2 based on the survey data of the main construction site containing gravel soil, and obtain the geometric parameters of various gravel soils from the PSD curve. Geometric parameters include d₁₀, d₃₀, d₆₀, d₇₀, d₈₅, d_{q}, wherein oho is the particle size of the particle whose loss of soil particles accounts for 10% of the total soil mass; d₃₀, d₆₀, d₇₀, d₈₅ have the same meaning with d₁₀, and d_{q} is the dividing diameter of the coarse and fine particles of the two types of gravel soil. Geometric parameters are substituted into

$${C}_{α} = \frac{{d}_{30}}{{d}_{60}}$$

to calculate the uneven coefficient Cᵤ of gravel soil, substituted into

$${C}_{α} = \frac{{d}_{n}^{2}}{{d}_{30} \times {d}_{60}}$$

to calculate the curvature coefficient C_{c}, and substituted into d_{q}=\sqrt{d₁₀d₁₀} to calculate d_{q}, so as to determine the boundary diameter of coarse and fine particles d_{q}, and obtain the corresponding fine particle content Pₓ in FIG. 2.

TABLE 1
Geometric Parameters of Three Different Soil Types Based On the PSD Curve

| | | Coefficient of | Curvature | | | | | | | |
|---|---|---|---|---|---|---|---|---|---|---|
| Soil | | Nonuniformity | Coefficient | Various Geometric Parameters (mm) | | | | | | |
| Type | Names | (Cᵤ) | (C_{c}) | d_{q} | d₁₀ | d₃₀ | d₆₀ | d₇₀ | d₁₅ | d₈₅ |
| Sand | Fine Sand S1 | 3.77 | 0.77 | 0.16 | 0.065 | 0.11 | 0.245 | 0.41 | 0.08 | 0.82 |
| | Gravel Soil S2 | 80.9 | 7.86 | 2.3 | 0.23 | 5.8 | 18.6 | 23.0 | 0.58 | 28.9 |
| | (Pebble*) | | | | | | | | | |
| Gravel | Gravel Soil S3 | | | | | | | | | |
| Soil | (Containing | 1141.3 | 0.047 | 0.57 | 0.015 | 0.11 | 17.1 | 21.6 | 0.022 | 28.3 |
| | Silty Clay | | | | | | | | | |
| | Pebbles*) | | | | | | | | | |

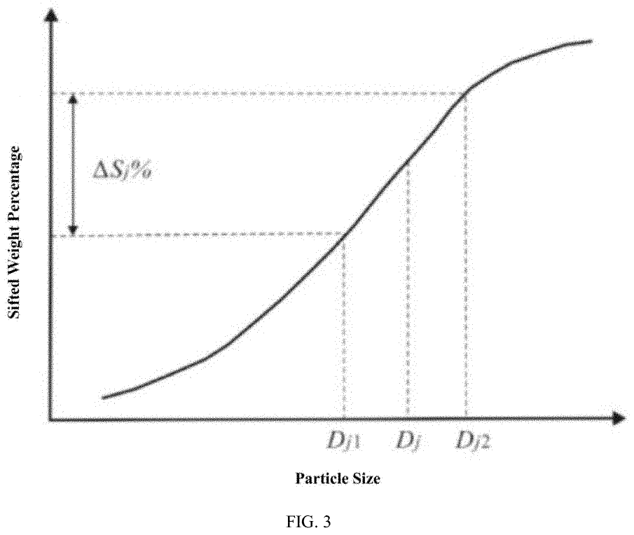

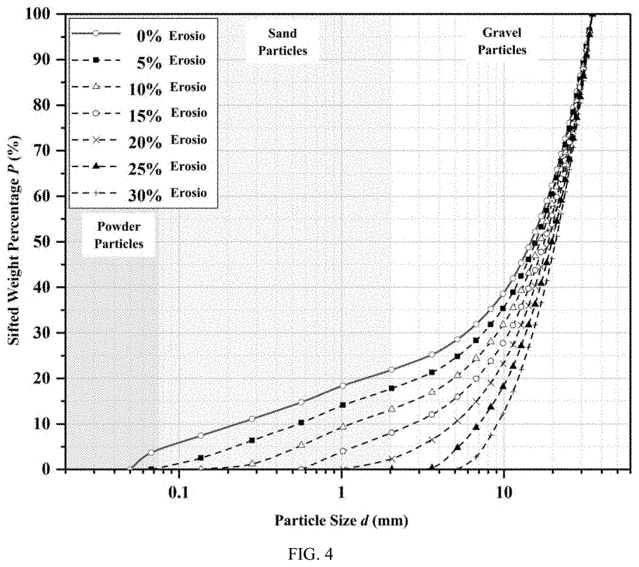

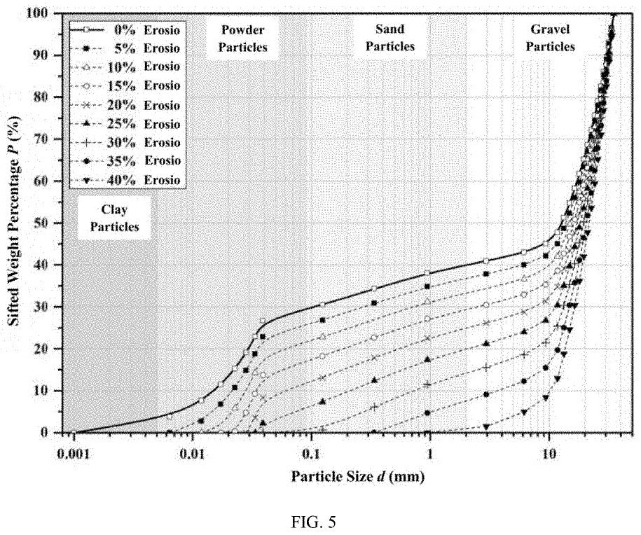

Calculate the soil particle content P and the soil porosity n of each grade of particle size according to the principle of graded erosion, as shown in FIG. 4 and FIG. 5, and draw the PSD curve of each grade of particle size according to the soil particle content P in each grade and the PSD curve cluster of the two gravel soils moving to the right under the graded erosion condition; each curve corresponds to the fine particles being eroded away step by step, and the composition of the soil particles gradually becomes simplified and single, and finally close to the new soil gradation of pebbles; from the comparison of the gradation curves of the two gravel soils (FIG. 3) and the moving PSD curve clusters (FIGS. 4 and 5), it can be seen that the initial state of the gravel soil S2 (the PSD curve with 0% erosion degree) is equivalent to the fifth grade erosion state of S3 (the PSD curve with 25% erosion degree). Therefore, it can be seen that the two gravel soils have similar sources of sedimentary materials, and the component structure of the former is the phased product of the latter after long-term hydraulic erosion.

calculate the soil particle content P and the soil porosity n of each grade of particle size according to the following formula (1):

$$\begin{matrix}{P}_{j}^{( i + 1 )} = \{ \begin{matrix}\frac{{P}_{j}^{( i )} - S}{100 - S} \times 100 \% & ( S \leq {P}_{j}^{( i )} < {P}_{x} ) \\ 0 & ( {P}_{j}^{( i )} < S < {P}_{x} )\end{matrix} & ( 1 a ) \\ {n}^{( i + 1 )} = {n}^{( i )} + ( 1 - {n}^{( i )} ) S & ( 1 b )\end{matrix}$$

As shown in FIG. 3, draw the PSD curve of each grade of particle size according to the soil particle content P in each grade; obtain the average particle diameter Dⱼ between the size grade from j₁ to j₂ from the figure, determine the equivalent diameter Dₕ of the soil particle according to the average particle diameter D₁, and calculate the minimum equivalent pore diameter d₀ according to the equivalent diameter Dₕ.

calculate the equivalent diameter Dₕ of the soil particle according to the following formula (2):

$${D}_{h} = \frac{1}{Σ \frac{Δ {S}_{j}}{{D}_{j}}} \qquad ( 2 )$$

calculate the minimum equivalent pore diameter d₀ according to the following formula (3):

$${d}_{0} = 2.67 \frac{n}{1 - n} \frac{{D}_{h}}{α} \qquad ( 3 )$$

calculate the critical hydraulic gradient i_{cr} of particle erosion at each stage according to the soil particle content P;

calculate the critical hydraulic gradient i_{cr} of particle erosion at each stage according to the following formula (4):

$${( {i}_{α} )}_{j} = \frac{0.85 {d}_{j}}{{P}_{j} {d}_{85}} ( 1 - n ) ( s - 1 ) \qquad ( 4 )$$

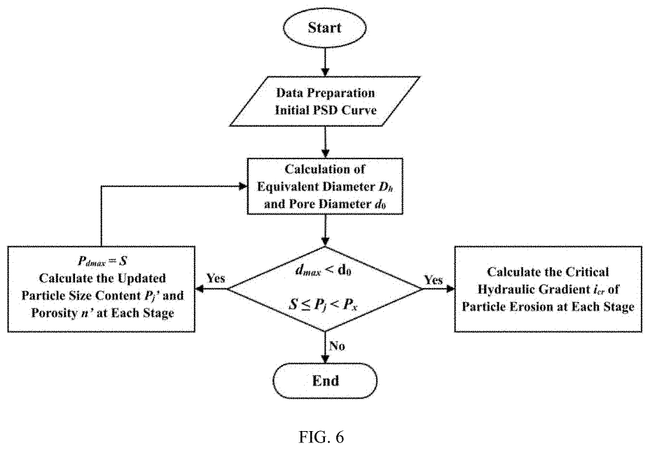

As shown in FIG. 6, through alternate loop iterations between Pⱼ⁽ⁱ⁾ and pⱼ⁽ⁱ⁺¹⁾, it is determined whether the continuous erosion condition is met after each iteration at the same time, so as to achieve a series of updated PSD curve clusters and the critical hydraulic gradient i_{cr} of particle erosion at each stage.

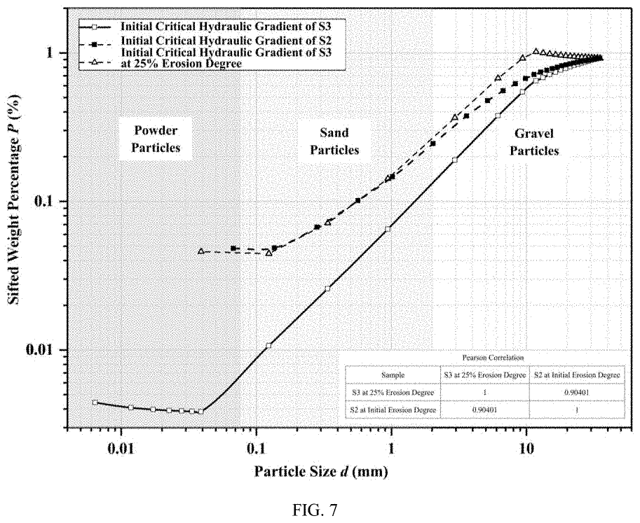

Draw a diagram showing the relationship between the critical hydraulic gradient and each grade of particle size of gravel soil as shown in FIG. 7 according to the critical hydraulic gradient i_{cr} of particle erosion at each stage; in FIG. 7, the critical hydraulic gradient i_{cr} of the gravel soil S2 is greater than the critical hydraulic gradient i_{cr} of S3; this is due to its small particle size range and large particle size, and the movement of soil particles requires greater permeability; in addition, under 25% erosion degree, the PSD curve of the gravel soil S3 is taken as the critical hydraulic gradient i_{cr} calculated by the new soil type as the change of the particle size, which is very similar to the initial critical hydraulic gradient i_{cr} change of the gravel soil S2, and the Pearson correlation coefficient reaches 90.4%. It can be further confirmed that the two gravel soil sedimentary materials have the same homology, and the former is the phased product of the latter after long-term hydraulic erosion.

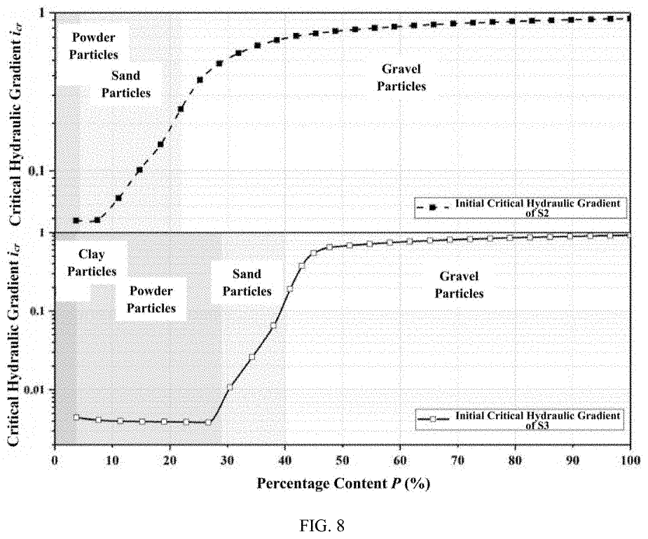

Draw a diagram showing the relationship between the critical hydraulic gradient of gravel soil and the percentage of particles as shown in FIG. 8 according to the critical hydraulic gradient i_{cr} of particle erosion at each stage; in the figure, the gravel soil S2 uses sand as the main component of erodible fine particles; while S3 uses powder and a small amount of clay as the main component of erodible fine particles, and the amount of sand that can be eroded is relatively small.

Calculate the permeability coefficient kₕ according to the geometric parameter values of each soil type and the minimum equivalent pore diameter do.

The minimum equivalent pore diameter d₀ is calculated by formula (2) and formula (3) and the geometric parameters are obtained directly from the PSD curve, and then substituted into formula (5) to obtain the permeability coefficient kₕ of gravel soil:

$${k}_{k} = 3.5 \times {10}^{- 4} \frac{{e}^{3}}{1 + e} \frac{{γ}_{w}}{{μ}_{w}} {d}_{10}^{2.32} {C}_{α}^{0.6} \qquad ( 5 )$$

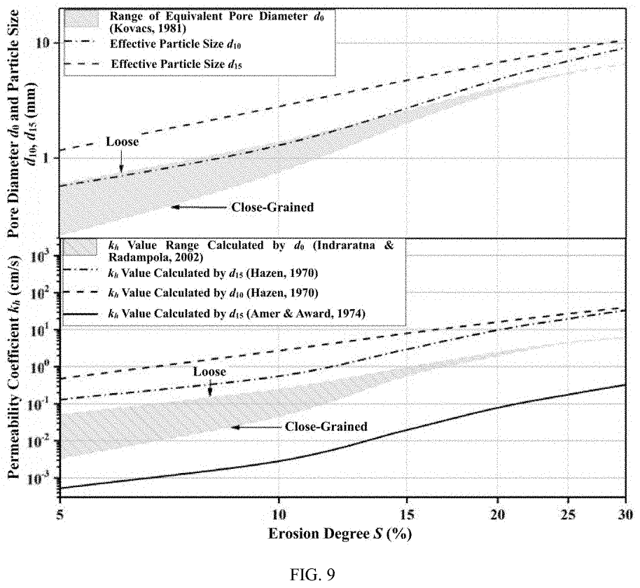

FIG. 9 is a diagram showing the relationship between geometric parameters and permeability coefficient of the gravel soil S2 under different erosion degrees; the data is read from the moving PSD curve or the geometric parameters calculated by the formula. For example, the particle size d₁₀ and d₁₅ can be read directly, and the pore diameter d₀ needs to be calculated by formula (3). It can be seen from FIG. 9 that taking into account the influence of the porosity n value (0.20-0.47), the filling area represents the range of geometric parameters and permeability coefficient under the influence of the porosity n value. As the erosion degree S increases, when S=20%, the geometric parameters or permeability coefficients will tend to be consistent.

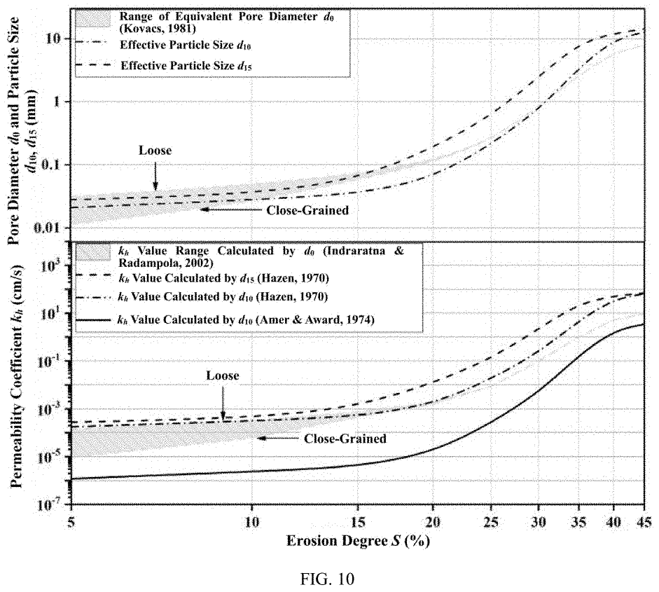

FIG. 10 is a diagram showing the relationship between geometric parameters and permeability coefficient of the gravel soil S3 under different erosion degrees; regardless of whether the effective particle size d₁₀ or d₁₅ is used, the value of the permeability coefficient is in the case of the erosion degree S<15%, and the calculation result is consistent with the result of the classical hydraulic formula. Taking into account the influence of the coefficient of nonuniformity Cᵤ in the formula (5), the permeability varies widely in the order of magnitude, and the influence of fine particles is highlighted. For example, the permeability coefficient of the gravel soil S3 ranges from 10⁻⁶ to 10 cm/s, which can cover the range of permeability coefficient from silt to gravel.

Calculate seepage flow velocity ν and total seepage flow Q according to Darcy law and the calculation formula of soil seepage flow rate;

calculate the seepage velocity ν of the soil particle according to the following formula (6):

v=Kₕ·i_{cr} (6)

calculate the total seepage flow Q of the soil particle according to the following formula (7):

Q=n·ν·A (7)

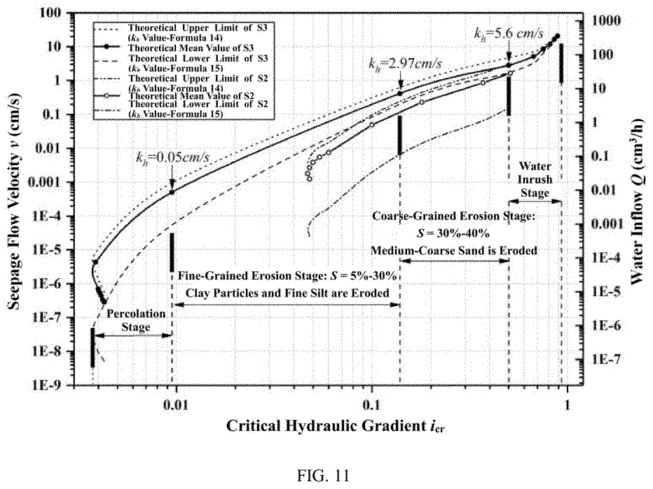

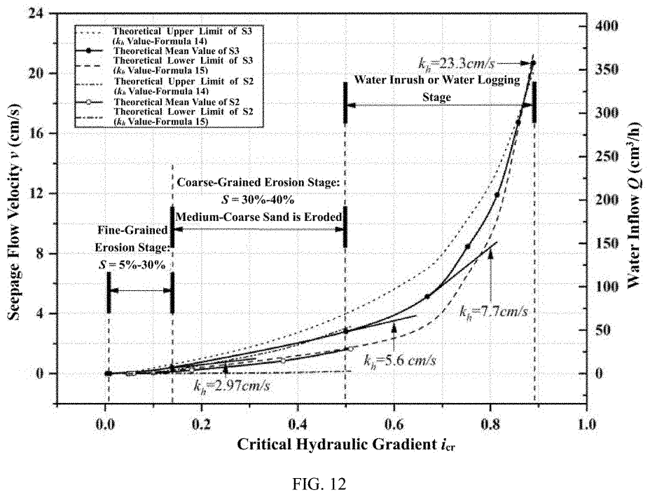

According to FIG. 11 and FIG. 12, as shown in Table 2, the hydraulic erosion properties of gravel soil at each erosion stage are obtained.

In the percolation stage (S<5%), the critical hydraulic gradient is i_{cr}<0.01, the permeation velocity ν is less than 5.0×10⁻⁴ cm/s, and the erodible particles are fine powder particles with a diameter of less than 0.01 mm;

In the fine-grained erosion stage (5% S [symbol] 30%), the critical hydraulic gradient i_{cr} is 0.01-0.13, and the seepage velocity ν=5.0×10⁻⁴−4.0×10⁻¹ cm/s; the erodible particles range from powder particles to fine sand particles with a diameter of 0.01-0.1 mm;

In the coarse-grained erosion stage (30% S [symbol] S [symbol] 40%), the critical hydraulic gradient i_{cr} is 0.13-0.50, and the seepage velocity ν=0.4-2.8 cm/s; the erodible particles are fine to medium coarse sand particles with a diameter of 0.1-0.57 mm;

In the water inrush or water logging stage (S is not taken into account), the critical hydraulic gradient i_{cr} is 0.50-0.89, and the seepage velocity is ν>2.8 cm/s.

TABLE 2
Parameters and Value Ranges of Gravel Soil Erosion at Each Stage

| | | | | Water |
|---|---|---|---|---|
| | | | | Inrush or |
| | | | | Water |
| Range of | | | | Logging |
| Parameter | | | | Stage |
| Values for | | | | S Is Not |
| Each Stage | Percolation | Fine-Grained | Coarse-Grained | Taken |
| of | Stage | Erosion Stage | Erosion Stage | Into |
| Erosion | S < 5% | 5% ≤ S < 30% | 30% ≤ S ≤ 40% | Account |
| Critical | <0.01 | 0.01-0.13 | 0.13-0.50 | >0.50 |
| Hydraulic | | | | |
| Gradient i_{cr} | | | | |
| Permeability | 0.05 | 0.05-2.97 | 2.97-5.6 | >5.6 |
| Coefficient | | | | |
| kₕ (cm/s) | | | | |
| Seepage | <5.0 × 10⁻⁴ | 5.0 × 10⁻⁴ − 0.4 | 0.4-2.8 | >2.8 |
| Flow | | | | |
| Velocity v | | | | |
| (cm/s) | | | | |
| Flow Rate | <8.6 × 10⁻³ | 8.6 × 10⁻³ − 6.9 | 6.9-48.4 | >48.4 |
| Per Unit | | | | |
| Area | | | | |
| Q*(m³/h) | | | | |

Note:
1: the porosity n is 0.47, that is, the gravel soil is in a loose state; A is 1.0 m².
2: With reference to the size of water inrush flow in domestic mines, when the flow is less than 50.0 m³/h, it is a small-scale water inrush point.

In summary, the method of the invention calculates the dynamic geometric parameters and the changed critical hydraulic gradient and permeability coefficient through the moving PSD curve under the condition of gravel soil graded erosion, and then calculates the seepage velocity and the water inflow by the Darcy formula, so as to obtain the rock and soil hydraulic characteristic parameters and the water inrush, which makes it possible to calculate the total seepage flow in the event of seepage erosion, and inversely deduce the degree of gravel soil erosion and dangerous conditions, so that corresponding measures can be taken to control and protect them, so as to avoid accidents. It is worthy of promotion.

Although the preferred embodiments of the invention have been described, however, those skilled in the art can make additional alterations and modifications to these embodiments once they learn the basic creative concept. Therefore, the appended claims are intended to be interpreted as including the preferred embodiments and all alterations and modifications falling within the scope of the invention.

Obviously, those skilled in the art can make various alterations and modifications to the invention without departing from the spirit and scope of the invention. In this way, if these alterations and modifications of the invention fall within the scope of the claims of the invention and the equivalent technologies thereof, the invention is also intended to include these alterations and modifications.

1. A method for determining hydraulic parameters and water inflow in the erosion stage of gravel soil, comprising the following steps:
calculate the soil particle content P and the soil porosity n of each grade of particle size according to the principle of graded erosion, and draw the PSD curve of each grade of particle size and the soil particle content P of each grade of particle size and the PSD curve cluster of each grade of particle size and the soil particle content P of each grade of particle size in each erosion stage;
calculate the equivalent diameter Dₕ of the soil particle according to the average particle diameter Dⱼ in a certain two particle size range in the PSD curve, and calculate the minimum equivalent pore diameter d₀ of the soil particle according to the equivalent diameter Dₕ;
calculate the critical hydraulic gradient i_{cr} of particle erosion at each stage according to the soil particle content P of each grade of particle size;
calculate the permeability coefficient kₕ according to the soil porosity n and the geometric parameter values of the soil particle in the PSD curve cluster;
calculate the seepage flow velocity ν and the total seepage flow Q according to the permeability coefficient kₕ.

2. The method for determining hydraulic parameters and water inflow in the erosion stage of gravel soil according to claim 1, wherein the method for calculating the soil particle content P and the soil porosity n of each grade of particle size is:
calculate the soil particle content Pⱼ⁽ⁱ⁾ of the j-th grade of particle size in the (1+1) state and the soil porosity n⁽ⁱ⁺¹⁾ updated to the (1+1) state according to the following formula (1):
$$\begin{matrix}{P}_{j}^{( i + 1 )} = \{ \begin{matrix}\frac{{P}_{j}^{( i )} - S}{100 - S} \times 100 \% & ( S \leq {P}_{j}^{( i )} < {P}_{x} ) \\ 0 & ( {P}_{j}^{( i )} < S < {P}_{x} )\end{matrix} & ( 1 a ) \\ {n}^{( i + 1 )} = {n}^{( i )} + ( 1 - {n}^{( i )} ) S & ( 1 b )\end{matrix}$$
in the formula, Pⱼ⁽ⁱ⁾ is the soil particle content of the j-th grade of particle size in the state (i); nⁱ is the soil porosity in the state (i); S is the degree of osmotic erosion, that is, the percentage of the mass of the soil particle smaller than a certain size that are washed away and eroded to the mass of the original soil particle; Pₓ is the content of fine particles, that is, the percentage of soil particles loss in the total soil mass.

3. The method for determining hydraulic parameters and water inflow in the erosion stage of gravel soil according to claim 1, wherein the method for calculating the minimum equivalent pore diameter d₀ of the soil particle according to the equivalent diameter Dₕ is:
calculate the equivalent diameter Dₕ of the soil particle according to the following formula (2):
$${D}_{h} = \frac{1}{Σ \frac{Δ {S}_{j}}{{D}_{j}}} \qquad ( 2 )$$
calculate the minimum equivalent pore diameter d₀ according to the following formula (3):
$${d}_{0} = 2.67 \frac{n}{1 - n} \frac{{D}_{h}}{α} \qquad ( 3 )$$
in the formula, Dⱼ is the average particle size of the soil particle with a size grade between j₁ and j₂; ΔSⱼ is the ratio of the weight of the j-th grade of particle size to the total weight of the sample; n is the porosity; α is the shape factor of the particle.

4. The method for determining hydraulic parameters and water inflow in the erosion stage of gravel soil according to claim 1, wherein the method for calculating the critical hydraulic gradient i_{cr} of particle erosion at each stage is:
calculate the critical hydraulic gradient (i_{cr}) j of the j-th grade of particle erosion according to the following formula (4):
$${( {i}_{α} )}_{j} = \frac{0.85 {d}_{j}}{{P}_{j} {d}_{85}} ( 1 - n ) ( s - 1 ) \qquad ( 4 )$$
in the formula, (i_{cr}) j is the critical hydraulic gradient of the j-th grade of particle erosion; s is the relative density, that is, the density of the overall soil density relative to the density of the water body; d₈₅ is the particle size of the particle whose loss of soil particles accounts for 85% of the total soil mass; dⱼ is the j-th grade of particle size that is eroded from the soil; pⱼ is the particle content of the j-th grade of particle size.

5. The method for determining hydraulic parameters and water inflow in the erosion stage of gravel soil according to claim 1, wherein the method for calculating the permeability coefficient kₕ is:
calculate the permeability coefficient kₕ according to the following formula (5):
$${k}_{k} = 3.5 \times {10}^{- 4} \frac{{e}^{3}}{1 + e} \frac{{γ}_{w}}{{μ}_{w}} {d}_{10}^{2.32} {C}_{α}^{0.6} \qquad ( 5 )$$
in the formula, e is the void ratio, calculated from the soil porosity e=n/(1−n); μ_{w} is the dynamic viscosity coefficient of water; γ_{w} is the weight of water; d₁₀ is the particle size of the particle whose loss of soil particles accounts for 10% of the total soil mass; Cᵤ is the coefficient of nonuniformity.

6. The method for determining hydraulic parameters and water inflow in the erosion stage of gravel soil according to claim 1, wherein the method for calculating the seepage flow velocity ν and the total seepage flow Q is:
calculate the seepage velocity ν of the soil particle according to the following formula (6):
v=Kₕ·i_{cr} (6)
calculate the total seepage flow Q of the soil particle according to the following formula (7):
Q=n·ν·A (7)
in the formula, ν is the seepage velocity; i_{cr} is the critical hydraulic gradient; Q is the total seepage flow; A is the area; n is the soil porosity.
